(19) United States
(12) Patent Application Publication
(10) Pub. No.: US 2026/0053578 A1
(43) Pub. Date: Feb. 26, 2026

(54) METHOD FOR MOUNTING AND/OR SECURING GUIDING ELEMENTS ON A SPATIALLY ADJUSTABLE DISK BY MEANS OF AT LEAST ONE CLAMPING ELEMENT, GUIDING ELEMENTS FOR MOVING A DISTAL-SIDE JOINT MECHANISM, MEDICAL INSTRUMENT, AND ROBOT

(71) Applicant: KARL STORZ SE & Co. KG, Tuttlingen (DE)

(72) Inventors: Dominik LÄNGLE, Tuttlingen (DE); Janosz SCHNEIDER, Tuttlingen (DE)

(73) Assignee: KARL STORZ SE & Co. KG, Tuttlingen (DE)

(21) Appl. No.: 19/104,211

(22) PCT Filed: Aug. 16, 2023
(86) PCT No.: PCT/EP2023/072507

§ 371 Date: Feb. 15, 2025

(30) Foreign Application Priority Data
Aug. 17, 2022 (DE) 10 2022 120 810.9

(51) Int. Cl. A61B 34/30; A61B 34/00

(52) U.S. Cl. CPC A61B 34/30; A61B 34/71; A61B 2034/301

(57) ABSTRACT

Provided are guiding elements, a medical instrument, a robot, and a method for mounting and/or securing guiding elements on a spatially adjustable disk, wherein the spatially adjustable disk has, at least partially, an internal cavity, an outer surface, an inner surface and a deflection contour for deflecting the guiding elements. The method includes: passing the guiding elements through a corresponding hole in the spatially adjustable disk, so that each passed-through portion of the guiding elements is arranged with the corresponding guiding element end on a proximal side of the spatially adjustable disk, guiding the passed-through portions of the guiding elements at least partially along the outer surface, the deflection contour and/or the inner surface of the spatially adjustable disk, and clamping the passed-through portions of the guiding elements to the outer surface, the deflection contour and/or the inner surface of the spatially adjustable disk.

### CROSS-REFERENCE TO RELATED APPLICATIONS

This application is the U.S. national stage of PCT/EP2023/072507 filed on Aug. 16, 2023which claims priority of German Patent Application No. 10 2022 120 810.9 filed on Aug. 17, 2022the contents of which are incorporated herein.

### TECHNICAL FIELD

The disclosure relates to a method for mounting and/or securing guiding elements on a spatially adjustable disk, wherein a distal-side joint mechanism for bending a distal end portion of a medical instrument can be moved by means of the guiding elements, and the spatially adjustable disk has, for each guiding element, a hole with a cross section through a thickness of the spatially adjustable disk, at least partially an internal cavity, an outer surface, an inner surface and a deflection contour for deflecting the guiding wires. Furthermore, the disclosure relates to guiding elements for moving a distal-side joint mechanism for bending a distal end portion of a medical instrument, to a medical instrument, and to a robot.

### BACKGROUND

Bendable instruments are often used in medical and non-medical applications and can be designed as manually guided and/or robotic instruments. In order to allow the shank of a medical instrument to be bent, four or more external guiding wires and/or guiding cables are usually arranged around pivoting members of a distal-side joint mechanism. For precise and homogeneous control of the distal, bendable end of the shank and/or the robot arm, the use of a plurality of thin guiding wires is advantageous in order to allow uniform movement and distribution of force in all bending directions. For this purpose, the guiding wires must be fixed in a tensioned state on the distal side (remote from the user) and on the proximal side (near the user).

US 2017/0281296 A1 discloses a medical instrument with guiding cables for moving an end effector, in which the guiding cables are guided through openings in a gimbal plate and a locking plate, which each have locking wedges on the mutually opposing sides, which locking wedges clamp the guiding cables by sliding when the gimbal plate and the locking plate are placed against one another. For fixing, the gimbal plate and the locking plate are then screwed to one another. The disadvantage here is that manufacturing tolerances during manufacture of the gimbal plate and the locking plate mean that the tensions with which the guiding cables are fixed can vary. There is also a risk that the guiding cables could be damaged by sharp edges on the gimbal plate and the locking plate and/or by the clamping. This results in the disadvantage that the individual strands of the guiding cable fray, thus significantly disrupting or complicating mounting. Due to the necessary handling of multiple components and the screw connections, mounting is very complex.

It is also known to screw the guiding wires to a proximal-side, drivable swashplate. To do this, each guiding wire must first be pre-tensioned individually and then screwed to the swashplate. In addition to being time-consuming, this can also lead to damage to the guiding wires. Furthermore, the threaded holes on the swashplate and the corresponding screws, which are usually inserted into and attached to the radially circumferential side wall of the swashplate, require a lot of installation space.

### SUMMARY

The object of the disclosure is to improve upon the prior art.

The object is achieved by a method for mounting and/or securing guiding elements on a spatially adjustable disk, wherein a distal-side joint mechanism for bending a distal end portion of a medical instrument can be moved by means of the guiding elements, and the spatially adjustable disk has, for each guiding element, a hole with a cross section through a thickness of the spatially adjustable disk, at least partially an internal cavity, an outer surface, an inner surface and a deflection contour for deflecting the guiding elements, comprising the following steps:
passing the guiding elements by means of their respective guiding element ends through respective holes in the spatially adjustable disk, so that each passed-through portion of the guiding elements is arranged with the corresponding guiding element end on a proximal side of the spatially adjustable disk,
guiding the passed-through portions of the guiding elements at least partially along the outer surface, the deflection contour and/or the inner surface of the spatially adjustable disk, and
clamping the passed-through portions of the guiding elements by means of at least one clamping element to the outer surface, the deflection contour and/or the inner surface of the spatially adjustable disk,
   so that the guiding elements are secured in a force-fitting manner on the spatially adjustable disk.

A method is thereby provided with which the guiding elements are quickly and easily mounted on a spatially adjustable disk, and the guiding element ends passed through the spatially adjustable disk are secured on the spatially adjustable disk by means of the at least one clamping element. It is particularly advantageous that the guiding elements are thereby connected in a tensioned state to the spatially adjustable disk in a force-fitting manner. Consequently, guiding elements are provided that are optimally fixed to the spatially adjustable disk with a fixed, predetermined tension in the tensioned state. By clamping by means of the at least one clamping element, the guiding elements are uniformly secured and thus homogeneously pre-tensioned. It is particularly advantageous that only one clamping element is required for this, and therefore all passed-through portions of the guiding elements can be clamped at the same time.

Consequently, a proximal drive movement on the spatially adjustable disk is optimally transmitted by means of the force-fittingly connected and tensioned guiding elements to the distal components of a distal joint mechanism for bending the distal end portion of the medical instrument.

Because the guiding elements are connected to the spatially adjustable disk in a force-fitting manner by means of the at least one clamping element, the materials of the connection partners can be freely selected. Thus, for example, the guiding elements can contain a nickel-titanium alloy, and the spatially adjustable disk can contain a chromium-nickel-molybdenum stainless steel. In contrast, an integral bond by means of welding would require that both the guiding wires and the spatially adjustable disk contain a nickel-titanium alloy. Consequently, the guiding wires can contain nitinol as a nickel-titanium alloy without the need for increased effort in forming the connection between the guiding elements and the spatially adjustable disk. In comparison with other materials, such as stainless steel, nitinol offers the advantage of being very elastic and very robust under alternating stress. The force-fitting connection allows quick and easy mounting without the guiding elements cutting into and/or settling in the joint mechanism after tensioning. It is particularly advantageous that the passed-through portions of the guiding elements are clamped flexibly by means of the at least one clamping element to the outer surface, the deflection contour and/or the inner surface of the spatially adjustable disk. Thus, both a flat and a contoured outer surface and/or inner surface and/or a specific deflection contour can be optionally used to guide and clamp the passed-through portions of the guiding elements. As a result, the passed-through portions of the guiding elements are pressed against the outer surface, the deflection contour and/or the inner surface of the spatially adjustable disk by means of the at least one clamping element, whereby the guiding elements are secured on the spatially adjustable disk due to the clamping force of the at least one clamping element such that they are firmly fixed in and/or on the spatially adjustable disk depending on the friction coefficient of the respective materials. Consequently, slipping of the guiding elements is prevented up to a defined load by clamping by means of the at least one clamping element. In addition to clamping, the tensile load on the guiding elements can also be increased by frictional forces and/or deflection along the outer surface, the deflection contour and/or the inner surface in a targeted manner by selection of the materials of the connection partners.

Since the passed-through portions of the guiding elements are guided at least partially along the outer surface, the deflection contour and/or the inner surface of the spatially adjustable disk and are clamped between the clamping element and the outer surface, the deflection contour and/or the inner surface, there is no or only a very small risk with the force-fitting connection that the strands of the guiding elements will fray and make mounting more difficult or even that a new guiding element must be guided and/or threaded through a hole in the spatially adjustable disk. Consequently, the mounting of the guiding elements within the joint mechanism is significantly simplified and can be carried out more quickly.

An essential concept of the disclosure is that the proximal guiding element ends are individually passed through the corresponding hole in the spatially adjustable disk and are guided on the proximal side of the spatially adjustable disk in a targeted manner at least partially along the outer surface, the deflection contour and/or the inner surface of the spatially adjustable disk, and the guided-along portions of the guiding elements are clamped by means of the at least one clamping element in a local position on the outer surface, the deflection contour and/or the inner surface to form a force-fitting connection, and the guiding elements are thereby secured on the spatially adjustable disk without further auxiliary materials and/or securing means being required in addition to the at least one clamping element. Depending on the shape of the outer surface, the inner surface and/or the deflection contour, a change in direction in the guidance of the guiding elements along the surface of the spatially adjustable disk can be utilized in a targeted manner, thereby improving the strength of the force-fitting connection and preventing slipping of the guiding elements. This means that friction between the materials of the guiding elements and the spatially adjustable disk as well as an areal transfer of the clamping force, which results from the guidance of the guiding elements along the outer surface, the deflection contour and/or the inner surface, to the spatially adjustable disk can both be used in a targeted manner for fixing in place.

The following terminology is explained:

A “guiding element” is in particular a thin and long, shaped flexible element. The elongate guiding element contains in particular a metal and/or a metal alloy. A guiding element can be a guiding wire and/or a guiding cable. A guiding wire is in particular a thin and long-shaped, flexible metal. A guiding element contains in particular a nickel-titanium alloy and thus nitinol, stainless steel and/or tungsten. Preferably, a guiding wire consists entirely of nitinol. The guiding wire in particular has a smooth surface. A guiding cable is an elongate, high-tensile element consisting of twisted or braided wires. Depending on the twisting or braiding, a guiding cable in particular has a structured surface. A guiding cable contains, for example, a stainless austenitic chromium-nickel-molybdenum steel (1.4401). In principle, a guiding element can have any cross-sectional shape, for example a circular, oval and/or curved cross section, a flat-edged, square or profiled wire cross section. Preferably, the guiding element has a round cross section. Typically, ≥3 or 4preferably 10 or any number of guiding elements are used in a joint mechanism inside the shank. In addition to the proximal-side securing of the guiding element ends on the spatially adjustable disk, the opposite distal guiding element ends are each fixed internally to the bendable distal end portion.

In the region of the distal bendable end portion, the guiding elements are in particular arranged running radially around the outside of pivoting members and/or member bodies, by means of which a finely articulated bending of the distal end portion is realized. A movement of the spatially adjustable disk caused by a proximal drive is transmitted via the guiding elements that are connected to the disk and are tensioned along the longitudinal direction of the shank as far as the distal guiding element ends fixed in the distal end portion, into a corresponding relative movement of the distal-side pivoting members, thus causing the distal end portion to bend. For fine motor control of the distal end portion of the medical instrument, in particular a plurality of thin guiding elements are used to achieve a more even distribution of force and thus relative movements in all possible bending directions.

A “joint mechanism” has in particular a “distal-side joint mechanism” and a “proximal-side joint mechanism.” The “proximal-side joint mechanism” has in particular the at least one clamping element, the spatially adjustable disk, associated shafts, and the proximal-side guiding element portions. On the distal side of the proximal-side joint mechanism, the guiding elements are in particular brought together, for example via a guide ring or a serrated washer, in the direction of the distal tip to a closer distance of the guiding elements from the longitudinal axis of the shank, so that they enter at the proximal end of the shank and are guided within the shank to the distal end portion substantially in parallel. For example, the diameter of the guiding elements radially surrounding the longitudinal axis of the shank is reduced from 18 mm to 4 mm. On the distal side of the proximal-side joint mechanism, the corresponding shaft of the spatially adjustable disk is in particular connected in the distal direction to a main shaft, via which the rotation of the shank can be realized. The proximal-side joint mechanism can be arranged in particular in the transition between the hand part and/or holding part of the medical instrument and the shank or in the hand part and/or holding part. The “distal-side joint mechanism” has in particular the distal-side guiding element portions and the pivoting members, by means of which bending of the distal end portion can be realized.

A “spatially adjustable disk” (also called a “swashplate”) is in particular a disk that is mounted such that, when moved by a proximal drive relative to the center of the disk, it executes an external pivoting movement transversely to the longitudinal axis through the disk and thus an up and down movement (wobble movement) on both sides. The swashplate in particular has a gimbal bearing. In order to transmit the respective proximal-side and distal-side movements, the spatially adjustable disk is connected in particular to a distal-side ball shaft or a distal-side ball joint and a proximal-side ball shaft or a distal-side ball joint. The guiding elements are guided by the thickness of the spatially adjustable disk. Thus, for example, when the shank is viewed in longitudinal cross section, the upper portion of the swashplate transverse to the longitudinal direction of the shank is displaced in the direction of the distal end, while the lower portion is displaced toward the proximal end, wherein the distal end portion is bent downward accordingly due to the corresponding movement of the guiding elements fixed to the swashplate. Consequently, in particular depending on the arrangement of the tensioned and fixed guiding elements on the swashplate, some guiding elements are pushed and other guiding elements are pulled simultaneously by the pivoting movement of the swashplate. The spatially adjustable disk contains in particular a stainless steel, a stainless steel alloy, aluminum and/or plastics material. The diameter of the spatially adjustable disk depends in particular on the desired bending angle of the distal-side joint mechanism. The swashplate can, for example, have a diameter in a range of 10 mm to 50 mm, in particular 15 mm to 40 mm, preferably 20 mm to 30 mm.

A “medical instrument” is in particular any mechanical or mechanical-electrical unit that is suitable for the diagnosis and/or treatment of humans or animals. The medical instrument is used in particular for the inspection of a human or animal body cavity and/or for the manipulation of human or animal tissue. The medical instrument has in particular a hand grip or hand part, a shank, and a tool and/or an optical system for viewing a field of vision. The medical instrument can in particular have a gripping tool, a cutting tool, a needle holder, a clip placement device, and/or another type of tool. A medical instrument is, for example, an endoscope with a long shank and a bendable end portion. The medical instrument can be a hand-held and/or manually guided instrument. The medical instrument can also be arranged as an end effector on a robot arm of a surgical robot and thus be a robot-assisted instrument. The medical instrument in particular has a flexible or rigid shank.

The “shank” of the medical instrument is in particular designed as an elongate tube. In particular, the shank has a diameter in a range of 2 mm to 10 mm. The shank can in particular have further components, such as an optical fiber for illuminating the object field, a working channel or multiple working channels for supplying rinsing fluid or a tool, such as a biopsy needle or an electrode. In particular in the case of a rigid shank, a central actuating element, for example a pull/push cable, for actuating a tool, for example a jaw part, on the bendable, distal instrument tip, can be arranged inside the shank from its proximal to distal end.

“Distal-side” and “distal” mean an arrangement and/or a corresponding end or portion that is close to the patient's body and therefore remote from the user. Accordingly, “proximal-side” or “proximal” mean an arrangement or a corresponding end or portion near the user and thus remote from the patient's body.

A “proximal drive” is a drive unit for influencing and thus pivoting the spatially adjustable disk. Thus, in particular, the drive movement of the proximal drive is transmitted into a pivoting movement of the spatially adjustable disk and, through the guiding elements secured on the spatially adjustable disk, to a corresponding relative movement of the distal-side pivoting members and/or member bodies in order to pivot the end portion of the medical device. The proximal drive can be a manual drive, for example based on the rotary movement of an actuating element, or a motorized drive. In the case of a motorized drive, it has one or more motors and/or a transmission, such as driven gears. The proximal drive is arranged in particular in the hand and holding part.

An “actuating unit” is in particular a component or consists of multiple components that act on the proximal drive. An actuating unit can in particular have one or more actuating elements. The actuating elements can be, for example, a push button or a rotary wheel, the movement of which is used to actuate the proximal drive. However, an actuating element of the actuating unit can also be an electronic control signal. Thus, the actuating unit can be a manually actuated handle or a module that is designed for robotic use and can be actuated without manual handling.

A “hole” is in particular a usually round opening through a thickness and/or a portion of the spatially adjustable disk. The hole in question can, for example, be formed by a guiding ring of the spatially adjustable disk. In particular, the hole is formed continuously substantially in the longitudinal direction of the shank and/or of a shaft of the proximal-side joint mechanism. The hole can be made in the spatially adjustable disk, for example, by means of laser cutting or water jet cutting, spark erosion or drilling. The holes can also be made directly during the manufacture of the spatially adjustable disk and thus during the shaping process, such as casting or sintering. However, the hole does not necessarily have to have a round cross section, but can also have a non-round shape or a shape adapted to the shape of the guiding element. Each hole has a cross section that is larger than the outer diameter of the guiding element passed through the corresponding hole.

A “clamping element” is in particular a component with which the proximal guiding element ends are connected to the spatially adjustable disk in a force-fitting manner. A clamping element is in particular any component that causes a clamping force and/or spring force and thereby clamps the portions of the guiding elements or the guiding element ends. Preferably, the portions of the guided guiding elements are clamped between a surface of the clamping element and a surface of the spatially adjustable disk. In principle, the clamping element can have any shape and/or any clamping mechanism. The clamping element serves in particular to form a detachable and/or force-fitting connection between the passed-through portions of the guiding elements and the spatially adjustable disk. A clamping element can be, for example, a screw clamp, a clamping block, a spring clamp or a circlip. A clamping element can be made up of one, two or more parts. For example, the clamping element can have two half rings with a tension spring arranged between them.

A “force-fitting connection” is in particular a detachable connection in which a normal force acts on the surfaces to be interconnected. The mutual displacement of the connected surfaces is prevented in particular as long as the counterforce caused by the static friction is not exceeded. The force-fitting connection between the corresponding guided portion of the guiding element and the swashplate is brought about in particular by means of the at least one clamping element.

The “outer surface” is in particular a surface or a portion of a surface on the outer side of the spatially adjustable disk. The “inner surface” is accordingly a surface or a portion of a surface on an inner side of the spatially adjustable disk. Thus, the inner surface is arranged on a side facing an enveloping space. In particular, the inner surface is a surface adjacent to the internal cavity of the spatially adjustable disk.

A “deflection contour” is in particular a region of the outline of the spatially adjustable disk that stands out from the surface of the spatially adjustable disk. The deflection contour has in particular a specific shape, such as a curvature, an S-shape and/or a rib shape. By means of the deflection contour, the direction of the guiding elements guided in the proximal direction is changed in particular. The guiding elements are guided in particular around the deflection contour and accordingly run around the outline of the deflection contour. The deflection contour can be formed and/or arranged on the outer surface, the proximal end, the end surface and/or on the inner surface of the spatially adjustable disk.

In a further embodiment of the method, the passed-through portions of the guiding elements are clamped by means of a second clamping element, a third clamping element and/or further clamping elements.

Thus, the passed-through portions of the guiding elements can be clamped to the spatially adjustable disk at different positions along the outer surface, deflection contour and/or inner surface by means of two or optionally more clamping elements, thus increasing the holding force and reducing the risk of the guiding elements slipping.

The second, third and/or further clamping element is, in terms of function, a clamping element as defined above. However, the clamping elements can have different shapes, clamping mechanisms and/or clamping forces.

In order to clamp all the passed-through portions of the guiding elements simultaneously as far as possible, a circlip or two or more circlips can be used as the at least one clamping element, the second clamping element, the third clamping element and/or as further clamping elements.

Consequently, all the guiding elements can be secured on the spatially adjustable disk at the same time by means of a single circlip.

A “circlip” (also called a “retaining ring”) is in particular a component for securing in place and thus for securing the passed-through portions of the guiding elements on the spatially adjustable disk. A circlip can in particular be an external circlip, which is placed in particular on a portion of the outer surface of the spatially adjustable disk and clamps the passed-through and guided-along portions of the guiding elements between its inner surface and the outer surface of the spatially adjustable disk. The circlip can also be an internal circlip, the outer side of which presses the portions of the guiding elements against the inner surface of the spatially adjustable disk. Accordingly, an external circlip and an internal circlip can also be used to clamp the portions of the guiding elements on the deflection contour depending on the position and/or shape of the deflection contour on the spatially adjustable disk. A circlip is in particular a standardized component. A circlip can also be a snap ring, a lamellar ring or a locking washer. Accordingly, circlip pliers are not required for the mounting and/or removal of the lamellar ring and the locking washer. Preferably, the circlip is positioned in a groove in the surface of the spatially adjustable disk. The force-fitting clamping and fixing of the portions of the guiding elements depends in particular on the corresponding clamping force of the circlip. Two or more circlips can differ from one another in their shape, diameter, material thickness and/or clamping force as well as other properties.

In a further embodiment of the method, the deflection contour is arranged at a proximal end of the spatially adjustable disk, and the passed-through portions of the guiding elements are deflected along and/or around the deflection contour of the spatially adjustable disk.

As a result of the arrangement of the deflection contour at the proximal end of the spatially adjustable disk, the passed-through portions of the guiding elements can first be guided in a proximal direction along the outer surface of the spatially adjustable disk and then guided in the opposite, distal direction along the inner surface of the spatially adjustable disk by means of the terminal, proximal-side deflection contour. Consequently, a change in the guide direction of the portions of the guiding elements of substantially 180° can be realized by means of the deflection contour. Because a deflection takes place and the portions of the guiding elements that are passed through and guided along the surface can have opposite directions on the outer surface and the inner surface, the clamped portions of the guiding elements are additionally prevented from slipping, and the guiding elements can be optimally fixed from the outer surface via the proximal end to the inner surface, both on the outside of the deflection contour and, with an encompassing design of the deflection contour, on the inside in the region of the cavity.

The clamping, the effect of the frictional force between the guiding elements and the spatially adjustable disk, and the change of direction due to the deflection thus achieve a fixed positioning and force-fitting connection of the portions of the guiding elements that have been passed through and guided along the spatially adjustable disk. These three mechanisms prevent the guiding elements from slipping even under high tensile and/or compressive loads when the proximal joint mechanism is actuated.

In order to position the clamping element and/or the circlip precisely on the spatially adjustable disk and/or to hold the proximal ends of the guiding elements in a precisely positioned manner, at least one groove is made in the outer surface, the deflection contour and/or the inner surface, and the clamping of the passed-through portions of the guiding elements by means of the clamping element and/or each clamping element takes place between a wall of the groove and the clamping element and/or each clamping element.

Thus, after the guiding elements have been passed through a corresponding hole in the spatially adjustable disk, and the passed-through portions of the guiding elements have been guided along, a clamping element and/or a circlip can be inserted into a preferably circumferential groove, whereby the guided guiding elements are pressed into this groove. In the process, the corresponding portion of the guiding elements is guided along the surface of the spatially adjustable disk and along the shape of the groove. By clamping, the guiding element portions are fixed locally in the groove such that they are firmly secured on and/or in the spatially adjustable disk depending on the clamping force of the clamping element and/or the spring force of the circlip and the friction coefficients of the materials of the guiding elements, the spatially adjustable disk, the clamping element and/or the circlip. Consequently, the force-fitting fixing of the guiding elements by the clamping element and/or the circlip in combination with a preferably circumferential groove increases the static friction of the guiding elements and thus prevents, up to a defined load, the guiding elements from slipping.

A “groove” is in particular an elongate depression in the surface of the spatially adjustable disk. A corresponding groove can be arranged in particular on the outer surface, the deflection contour or the inner surface of the spatially adjustable disk. The groove can in particular be circumferential, continuous or stepped. The groove can have a rectangular cross section, a trapezoidal shape, a dovetail shape and/or another shape. The clamping element and/or the circlip can be inserted and/or positioned into the groove. The ends of the guiding elements can be received in the groove and arranged on a wall of the groove. Depending on the position of the groove in the surface of the spatially adjustable disk, the groove can be inserted horizontally, vertically or obliquely into the corresponding surface of the spatially adjustable disk.

In a further embodiment of the method, the inner surface and/or the groove has at least one receiving element or one receiving element each for receiving the guiding element ends or a corresponding guiding element end.

Thus, the at least one receiving element or multiple receiving elements can be inserted into the groove in a targeted manner before the clamping element and/or the securing element are inserted. A receiving element can, for example, be a slot and thus a further depression in the groove or drilled holes in the wall of the groove, into which the respective guiding element ends are inserted before the clamping element and/or the circlip are inserted into the groove. Thus, the proximal ends of the guiding elements can either butt against a wall of the groove or are received in one or more receiving elements of the inner surface and/or the groove.

In order to utilize the entire radially circumferential outer surface and/or inner surface of the spatially adjustable disk for clamping, a swashplate is used as the spatially adjustable disk.

In a further embodiment of the method, in order to allow stress-free mounting and subsequent pre-tensioning, the spatially adjustable disk with a distal-side ball shaft is inserted into a main shaft of the proximal-side joint mechanism before clamping, and, after the force-fitting securing has been formed, the spatially adjustable disk with the distal-side ball shaft is partially pulled out of the main shaft in the proximal direction in order to pre-tension the force-fittingly secured guiding elements.

A “ball shaft” is in particular a shaft with a ball joint. The spatially adjustable disk (swashplate) has in particular a proximal-side ball shaft for transmitting the drive movement of the proximal drive to the spatially adjustable disk in the form of a pivoting movement. For this purpose, the swashplate has in particular a receptacle for sliding on the ball head of the ball shaft and thus a movable receptacle around all axes. Furthermore, the spatially adjustable disk has a distal-side ball shaft for pre-tensioning the fixed guiding elements on the spatially adjustable disk for connection to the main shaft and/or for transmitting the movement to the main shaft.

In a further aspect of the disclosure, the object is achieved by guiding elements for moving a distal-side joint mechanism in order to bend a distal end portion of a medical instrument, wherein the guiding elements are secured on a spatially adjustable disk by means of a method described above.

Thus, force-fittingly connected guiding elements are provided, which, when used in a shank in a medical instrument, allow a continuously variable and very smooth control of the distal joint mechanism in order to bend the distal end portion of the medical instrument.

Furthermore, a shank with guiding elements is provided, which are secured according to methods described above and allow an optimal, reliably controllable joint mechanism in the shank due to the reliable force-fitting connection by means of clamping, friction and/or deflection and the uniform, defined tension. In particular, the shank is detachably connectable to the hand part and/or holding part of a medical instrument and/or reusable and/or designed for single use.

In an additional aspect of the disclosure, the object is achieved by a medical instrument having an elongate shank, wherein an actuating unit for actuating a proximal drive is arranged at a proximal end of the shank, and an end portion is arranged at a distal end, and the end portion is bendable relative to a longitudinal axis of the shank by means of a distal-side joint mechanism, multiple guiding elements are passed through the elongate shank and connect the proximal-side drive to the distal-side joint mechanism, wherein the proximal-side drive can act on a spatially adjustable disk, and the guiding elements are passed with a corresponding proximal guiding element end through a corresponding hole through a thickness of the spatially adjustable disk, wherein portions of the guiding elements arranged proximally to the spatially adjustable disk are arranged along an outer surface, a deflection contour and/or an inner surface of the spatially adjustable disk and are connected in a force-fitting manner to the spatially adjustable disk by means of at least one clamping element.

In a further embodiment of the medical instrument, the guiding elements contain a nickel-titanium alloy, and the spatially adjustable disk and/or the at least one clamping element contain a material with a higher material hardness than the nickel-titanium alloy.

Preferably, at least the spatially adjustable disk has a higher material hardness than the guiding elements. The clamping element and/or the circlip can have a material hardness equal to or higher than the guiding elements. The circlip contains, for example, hardened steel. Likewise, the circlip and/or the clamping element can also comprise the same or a different nickel-titanium alloy as the guiding elements.

A “nickel-titanium alloy” is in particular a nickel-titanium intermetallic compound. A nickel-titanium alloy is in particular nitinol. “Nitinol” is in particular an intermetallic phase NiTi with an ordered, cubic crystal structure, which differs from that of titanium and nickel. The nickel-titanium alloy and nitinol usually have a slightly higher proportion of nickel, for example 55%, and titanium. However, nitinol can also contain 50% nickel and 50% titanium and/or other alloy ratios and/or additional alloy components in small amounts. In particular, nitinol exhibits thermal shape memory and superelasticity. Nitinol returns to its original shape, in particular after plastic deformation, when the nitinol is heated. This thermal shape memory and the mechanical shape memory as superelasticity are in particular caused by a thermoelastic, martensitic transformation in the solid state. Conventional methods of component manufacturing at room temperatures are usually not suitable due to the extreme elasticity of nitinol and/or its thermal shape memory. In principle, cutting processes for shaping are possible, but these are associated with considerable tool wear. In this respect, it is advantageous that if the guiding elements and the clamping element are both made of nitinol, the swashplate can be manufactured and cut from a different material. The guiding elements made of nitinol are produced in particular by drawing, wherein the wire is soft-annealed between drawing processes. Nitinol can be shaped, for example, by grinding or electroerosion. Due to the superelastic properties, the guiding elements made of nitinol can in particular be bent to a greater extent than guiding elements made of stainless steel, for example. In addition, the guiding elements made of nitinol can be bent multiple times, but still remain controllable. Furthermore, nitinol maintains its shape even under tension and is kink-resistant.

In a further embodiment, the medical instrument has a second clamping element, optionally a third clamping element and/or further clamping elements for the force-fitting securing of the guiding elements on the spatially adjustable disk, and/or a second deflection contour, optionally a third deflection contour and/or further deflection contours for deflecting the guiding elements.

Thus, two or more deflection contours can be arranged at different positions on the inner and/or outer surface of the spatially adjustable disk and have different shapes and/or material thicknesses in comparison with the surrounding surface of the spatially adjustable disk.

In order to clamp and secure the guiding elements radially and evenly around the outer surface, the inner surface and/or the deflection contour, the clamping element, each clamping element or the clamping elements is or are designed as an external circlip and/or as an internal circlip.

In a further embodiment of the medical instrument, the at least one deflection contour, each deflection contour or the deflection contours is or are a curved contour, a contour elevated above the surface of the spatially adjustable disk and/or a curved and/or thickened edge at a proximal end of the spatially adjustable disk.

Of course, a deflection contour can also be designed differently in some portions. For example, a deflection contour can initially be designed as a straight conical extension in the longitudinal direction of the guiding elements and have a subsequent portion with a curved contour or a terminal semicircular contour.

In a further aspect of the disclosure, the object is achieved by a robot with at least one robot arm for holding and/or positioning a medical instrument and/or with an actuator for controlling a distal-side joint mechanism of the medical instrument, wherein the medical instrument is a previously described medical instrument, so that the medical instrument can be positioned by means of the at least one robot arm, and/or the distal-side joint mechanism can be actuated by the action of the actuator of the robot on the proximal drive of the medical instrument.

Thus, the guiding elements secured on a spatially adjustable disk can be used not only in a manually guided medical instrument but also in a robotically guided instrument. By means of the robot, it is ensured that the medical instrument is held fixedly at the end of the at least one robot arm and that the medical instrument is oriented in a precisely positioned manner. On the other hand, the joint mechanism and the bending of the distal end portion of the medical instrument as an end effector can be guided very precisely via an input device on the robot, for example by means of a joystick on the control console and/or input grips of the robot that are secured on the hand. In principle, the proximal drive of the medical instrument can also be arranged in the distal end of the robot arm and a coupling point and/or interface between the distal end of the robot arm and the holding unit of the medical instrument can be designed accordingly.

A “robot” is a medical robot. A robot is in particular a surgical robot. The robot is typically a telemanipulator, which uses the surgeon's input elements and/or operating elements on one side to control the medical instrument as an end effector on the other side at the end of a robot arm. The robot preferably has multiple robot arms, wherein a camera, in particular a three-dimensional camera, is arranged on one robot arm, and one or more exchangeable medical instruments are arranged on the robot arm or the other robot arms. Each robot arm is in particular designed to be movable on 3 to 8 axes. Instead of one robot with multiple arms, it is of course possible to use multiple robots, each with one arm or even just two arms, which are controlled together. The camera can also be held endoscopically or exoscopically.

Through a coupling point and/or interface between the end of the robot arm holding the medical instrument and the medical instrument, the proximal drive of the medical instrument can be actuated by means of the actuator of the robot arm. Any drive component or module that converts an electrical signal into a mechanical movement can be used as an actuator.

Further embodiments, as well as some of the advantages associated with these and other embodiments, will become apparent and better understood from the following detailed description with reference to the accompanying figures. Objects or parts thereof that are substantially the same or similar may be provided with the same reference signs. The figures are only a schematic representation of an embodiment of the disclosure. The drawings show an exemplary embodiment of the disclosure. The drawings, the description, and the claims contain numerous features in combination. A person skilled in the art will also, expediently, consider the features individually and combine them into useful further combinations.

### BRIEF DESCRIPTION OF DRAWINGS

The disclosure is explained in more detail below with reference to exemplary embodiments. In the drawings:

FIG. 1 is a schematic, three-dimensional representation of connected guiding wires on a swashplate,

FIG. 2 is a schematic representation of a proximal-side joint mechanism with the swashplate, the connected guiding wires, and a main shaft,

FIG. 3 is a schematic, three-dimensional representation of the proximal-side joint mechanism from FIG. 2 in a distal-side view,

FIG. 4 is a three-dimensional representation of a video endoscope with a bendable tip of a shank,

FIG. 5 is a highly schematic representation of a surgical robot with an endoscopic instrument secured on a robot arm, and

FIG. 6 is a flow chart of a method for mounting and/or securing guiding wires.

### DETAILED DESCRIPTION OF PREFERRED EMBODIMENTS

A medical instrument 101 comprises a video endoscope 127, wherein the video endoscope 127 has a hand grip 133 and a flexible shank 129. Multiple external operating elements 135 for operating the video endoscope 127 by a user are arranged on the hand grip 133. Furthermore, the hand grip 133 of the video endoscope 127 is connected to a supply hose 137, at the end of which a plug 141 is arranged. The flexible shank 129 of the video endoscope 127 has a bendable tip 131, which can be bent by means of an internal joint mechanism in the shank 129 via the operating elements 135 (FIG. 4).

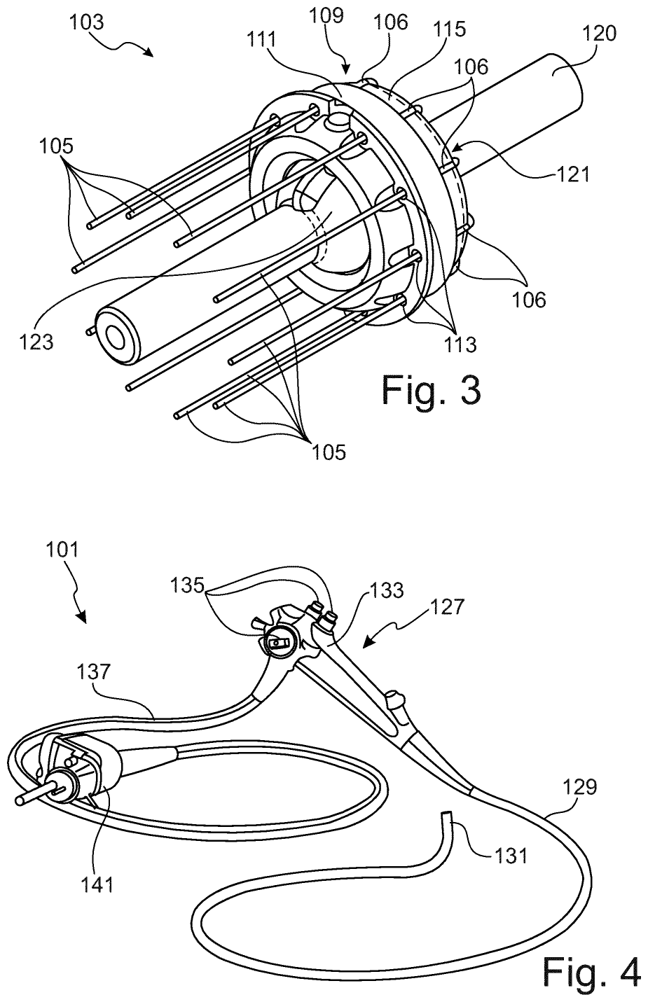

A joint mechanism of the shank 129 has an internal proximal-side joint mechanism 103, which has a swashplate 109 made of stainless steel with an external, integrated guiding ring 111. The guiding ring 111 has ten continuous through holes 113. The swashplate 109 has a cavity 110 on the inside. A main shaft 120 with a proximal-side ball shaft 121 and a distal-side ball shaft 123 is passed through the cavity 110 of the swashplate 109 along a longitudinal center axis 143 (see FIGS. 2 and 3). By means of a drive (not shown), a pivoting movement is imposed on the swashplate 109 via the proximal-side ball shaft 121. Furthermore, the joint mechanism has ten guiding wires 105 made of nitinol, which are secured internally in the region of the bendable tip 131 on the distal side and are guided in a tensioned manner through the shank 129 to the opposite proximal-side end of the shank 129 to the proximal-side joint mechanism 103.

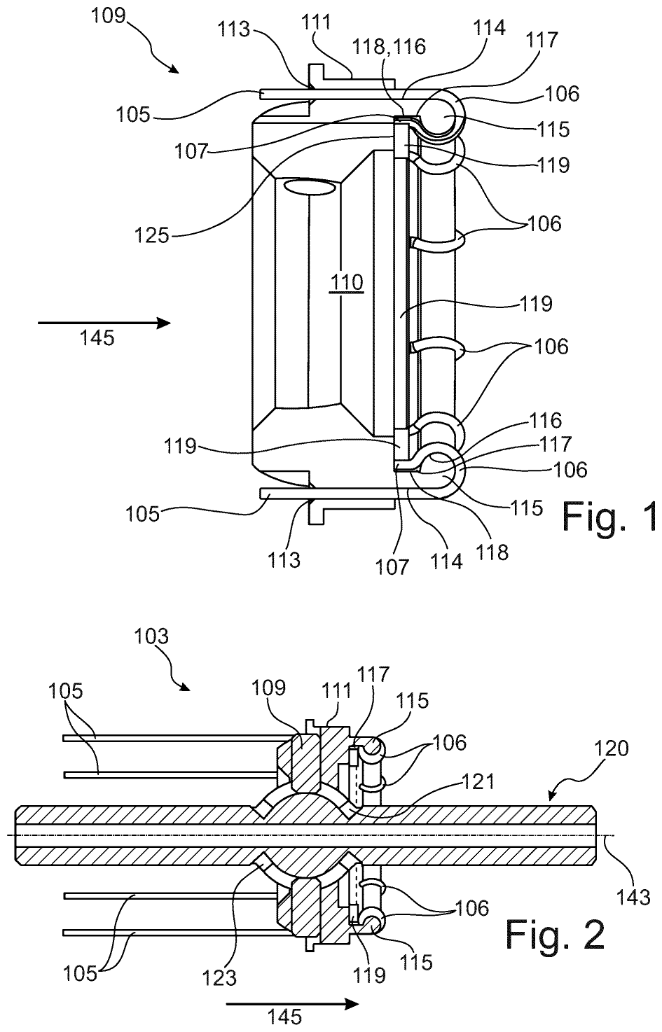

Furthermore, the swashplate 109 has an outer surface 114 on the proximal side of the guiding ring 111 and an encompassing, thickened deflection contour 115 at the proximal end. The deflection contour 115 merges into an inner surface 116 on the inside around the cavity 110. In the inner surface 116, a groove 117 is arranged with a wall 118, which is horizontally aligned in FIG. 1, and a vertical, proximal-side wall 125.

On the proximal side, the guiding wires 105 are guided in a manner distributed evenly radially all the way around through one through hole 113 each and further along the outer surface 114 in the proximal direction 145 around the deflection contour 115 and thereby guided back in the opposite direction in the distal direction, wherein the proximal guiding wire portions 106 guided in this way merge at the end into proximal guiding wire ends 107, which bear against the wall 118 of the groove 117 and butt against the proximal-side wall 125 of the swashplate 109 arranged vertically to the wall 118. A circlip 119 is inserted into the groove 117, with its outer surface clamping the proximal guiding wire ends 107 radially all the way around between its outer surface and the wall 118 of the groove 117.

To secure the guiding wires 105 made of nitinol on the swashplate 109 containing stainless steel, the following steps are carried out in a method 201 for mounting and/or securing guiding wires 105 (FIG. 6):

The respective proximal guiding wire portions 106 of the guiding wires 105 are each passed through a through hole 113 in the integrated guiding ring 111 of the swashplate 109, so that the respectively passed-through proximal guiding wire portions 106 are arranged on the proximal side of the swashplate 109 (step 203, FIG. 6). Subsequently, the passed-through guiding wire portions 106 are guided along the outer surface 114 of the swashplate 109 (step 205), and then the guiding wire portions are deflected around the terminal, thickened deflection contour 115 (step 207), so that the guiding wire portions 106 guided in the proximal direction 145 are guided on the inside around the cavity 110 in the opposite direction along the inner surface 114 and the wall 118 after being deflected by means of the deflection contour 115, and the proximal guiding wire ends 107 butt against the proximal-side wall 125 transversely to the longitudinal center axis 143. Subsequently, as a result of the insertion of the circlip 119 into the groove 117, the proximal guiding wire ends 107 are clamped radially all the way around between the outer side of the circlip 119 and the wall 119 (step 209), whereby the guiding wires 105 are secured on the swashplate 109 on the proximal side in a force-fitting and positionally secure manner.

In an alternative of the swashplate 109 (not shown in FIG. 2), an external circlip has additionally been placed on the proximal-side guiding wire portions 106 on the proximal side of the integrated guiding ring 111, thus repeating 211 the step 209 (FIG. 6).

In a further alternative, steps 203 to 209 have been carried out first, wherein an external circlip (not shown) has been placed, and then, after the deflection contour 115 with a complete deflection 207 in the distal direction, a repetition 211 takes place with a further guiding of the guiding wire portions 106 along the inner surface 114 and the wall 118 in the groove 117 (step 207) and the clamping of the guiding wire ends 107 in the groove by means of the circlip 119 (step 209).

In the case of a further alternative with a multi-part clamping element, steps 203 to 209 can also be repeated one after the other (step 211), so that the corresponding guiding wire 105 or two or more guiding wires 105, but not all ten guiding wires 105, are passed through the associated through hole 113 (203), guided along the corresponding surface 116, 114 of the swashplate 109 (205), deflected around the deflection contour 115 (207) and then secured by clamping the guiding wire portions 106 (step 209). Subsequently, all steps 203 to 209 are repeated one after the other for one guiding wire 105 or multiple guiding wires 105 of the guiding wires 105 not yet passed through (211, FIG. 6).

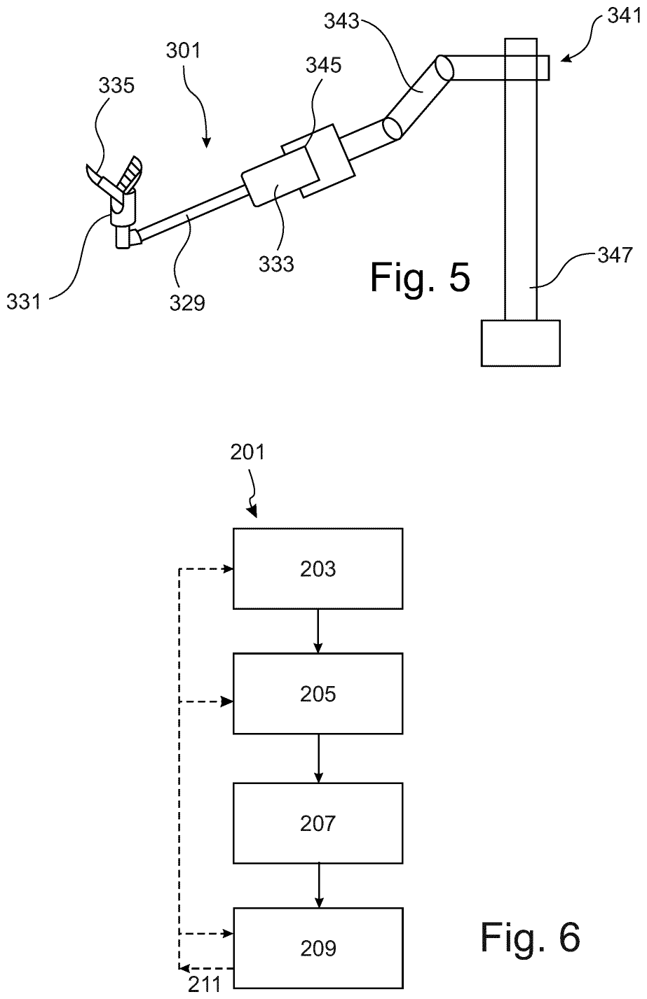

In an alternative of the medical instrument, an endoscopic instrument 301 is designed as an end effector of a surgical robot 341. The surgical robot 341 has a base 347 with four robot arms, wherein in FIG. 5 only the robot arm 343 that holds the endoscopic instrument 301 is shown. The proximal-side joint mechanism 103 shown in FIG. 2 is arranged in a shank 329 of the endoscopic instrument 301, wherein the endoscopic instrument 301 has at its distal end a bendable tip 331 with a terminal jaw tool 335 (the jaw tool 335 is not shown to scale in FIG. 5). At the proximal end of the shank 329, the endoscopic instrument 301 has a holding unit 333, which is held at the end of the robot arm 343 of the surgical robot 341. An interface 345 for coupling is arranged between the holding unit 333 of the endoscopic instrument 301 and the end of the robot arm 343, at which interface an internal proximal drive of the endoscopic instrument 301 is actuated via a likewise internal actuator (not shown in FIG. 5) of the surgical robot 341. As a result, a pivoting movement of the swashplate 109 is imposed via the proximal-side ball shaft 121, wherein, due to the force-fitting connection of the guiding wires 105 to the swashplate 109 and the tensioned state of the guiding wires 105, the induced pivoting movement is transmitted to the bendable tip 331 in a corresponding bending movement.

Thus, a medical instrument 101 with a video endoscope 127 and a surgical robot 341 with an endoscopic instrument 301 are provided, in which a continuously variable, homogeneous and smooth movement of the corresponding bendable tip 131, 331 is brought about by a force-fitting connection of the proximal guiding wire ends 107 and guiding wire portions 106 of the guiding wires 105 with the swashplate 109.

The drawings show an exemplary embodiment of the disclosure. The drawings, the description, and the claims contain numerous features in combination. A person skilled in the art will also, expediently, consider the features individually and combine them into useful further combinations. The disclosure relates to a method for mounting and/or securing guiding elements on a spatially adjustable disk, wherein the spatially adjustable disk has, at least partially, an internal cavity, an outer surface, an inner surface and a deflection contour for deflecting the guiding elements, comprising the following steps:
passing the guiding elements by means of their respective guiding element ends through a corresponding hole in the spatially adjustable disk, so that each passed-through portion of the guiding elements is arranged with the corresponding guiding element end on a proximal side of the spatially adjustable disk,
guiding the passed-through portions of the guiding elements at least partially along the outer surface, the deflection contour and/or the inner surface of the spatially adjustable disk, and
clamping the passed-through portions (106) of the guiding elements by means of at least one clamping element to the outer surface, the deflection contour and/or the inner surface of the spatially adjustable disk,
   so that the guiding elements are secured in a force-fitting manner on the spatially adjustable disk. The disclosure also relates to guiding elements, a medical instrument and a robot.

1. A method for mounting and/or securing guiding elements on a spatially adjustable disk, wherein a distal-side joint mechanism for bending a distal end portion of a medical instrument can be moved by means of the guiding elements, and the spatially adjustable disk has, for each guiding element, a hole with a cross section through a thickness of the spatially adjustable disk, at least partially an internal cavity, an outer surface, an inner surface and a deflection contour for deflecting the guiding elements, comprising the following steps:
passing the guiding elements by means of their respective guiding element ends through respective holes in the spatially adjustable disk, so that each passed-through portion of the guiding elements is arranged with the corresponding guiding element end on a proximal side of the spatially adjustable disk,
guiding the passed-through portions of the guiding elements at least partially along the outer surface, the deflection contour and/or the inner surface of the spatially adjustable disk, and
clamping the passed-through portions of the guiding elements by means of at least one clamping element to the outer surface, the deflection contour and/or the inner surface of the spatially adjustable disk, so that the guiding elements are secured in a force-fitting manner on the spatially adjustable disk.

2. The method according to claim 1, wherein the passed-through portions of the guiding elements are clamped by means of a second clamping element, a third clamping element and/or further clamping elements.

3. The method according to claim 1, characterized in that a circlip or two or more circlips is or are used as the at least one clamping element, the second clamping element, the third clamping element and/or as further clamping elements.

4. The method according to claim 1, wherein in that the deflection contour is arranged at a proximal end of the spatially adjustable disk and the passed-through portions of the guiding elements are deflected along and/or around the deflection contour of the spatially adjustable disk.

5. The method according to claim 1, wherein at least one groove is made in the outer surface, the deflection contour and/or the inner surface, and the clamping of the passed-through portions of the guiding elements by means of the clamping element and/or each clamping element takes place between a wall of the groove and the clamping element and/or each clamping element.

6. The method according to claim 1, wherein the inner surface and/or the groove has or have at least one receiving element or one receiving element each for receiving the guiding element ends or a corresponding guiding element end.

7. The method according to claim 1, wherein a swashplate is used as the spatially adjustable disk.

8. The method according to claim 1, wherein the spatially adjustable disk with a distal-side ball shaft is inserted into a main shaft of the proximal-side joint mechanism before clamping, and, after the force-fitting securing has been formed, the spatially adjustable disk with the distal-side ball shaft is partially pulled out of the main shaft in the proximal direction in order to pre-tension the force-fittingly secured guiding elements.

9. The method of claim 1 wherein the guiding elements for moving a distal-side joint mechanism in order to bend a distal end portion of a medical instrument, are secured on a spatially adjustable disk.

10. A medical instrument having an elongate shank, wherein an actuating unit for actuating a proximal drive is arranged at a proximal end of the shank, and an end portion is arranged at a distal end, and the end portion is bendable relative to a longitudinal axis of the shank by means of a distal-side joint mechanism, multiple guiding elements are passed through the elongate shank and connect the proximal-side drive to the distal-side joint mechanism, wherein the proximal-side drive can act on a spatially adjustable disk, and the guiding elements are passed with a corresponding proximal guiding element end through a corresponding hole through a thickness of the spatially adjustable disk, characterized in that portions of the guiding elements arranged proximally to the spatially adjustable disk are arranged along an outer surface, a deflection contour and/or an inner surface of the spatially adjustable disk and are connected in a force-fitting manner to the spatially adjustable disk by means of at least one clamping element.

11. The medical instrument according to claim 10, wherein the guiding elements contain a nickel-titanium alloy, and the spatially adjustable disk and/or the at least one clamping element contain a material with a higher material hardness than the nickel-titanium alloy.

12. The medical instrument according to claim 10, the medical instrument has a second clamping element, a third clamping element and/or further clamping elements for the force-fitting securing of the guiding elements on the spatially adjustable disk, and/or a second deflection contour, a third deflection contour and/or further deflection contours for deflecting the guiding elements.

13. The medical instrument according to claim 10, wherein the clamping element, each clamping element or the clamping elements is or are designed as an external circlip and/or as an internal circlip.

14. The medical instrument according to claim 10, wherein the at least one deflection contour, each deflection contour or the deflection contours is or are a curved contour, a contour elevated above a surface of the spatially adjustable disk and/or a curved and/or thickened edge at a proximal end of the spatially adjustable disk.

15. A robot with at least one robot arm for holding and/or positioning a medical instrument and/or with an actuator for controlling a distal-side joint mechanism of the medical instrument, wherein the medical instrument is the medical instrument according to claim 10, so that the medical instrument can be positioned by means of the at least one robot arm, and/or the distal-side joint mechanism can be actuated by the action of the actuator of the robot on the proximal drive of the medical instrument.
